(19) United States
(12) Patent Application Publication
(10) Pub. No.: US 2025/0204770 A1
(43) Pub. Date: Jun. 26, 2025

(54) SYSTEM AND METHOD FOR SUBJECTIVE FLOATER ASSESSMENT

(71) Applicant: Alcon Inc., Fribourg (CH)

(72) Inventor: Steven T. Charles, Memphis, TN (US)

(21) Appl. No.: 18/990,328

(22) Filed: Dec. 20, 2024

Related U.S. Application Data
(60) Provisional application No. 63/614,830, filed on Dec. 26, 2023.

(51) Int. Cl. A61B 3/024; G16H 50/20

(52) U.S. Cl. CPC A61B 3/024; G16H 50/20

(57) ABSTRACT

An automated system for assessing a subjective severity of a patient-perceived floater in an eye of a patient includes a display screen, a patient-operated feedback device, and a floater assessment system (FAS). The FAS is configured to present a visual target on the display screen at a primary fixation point, and possibly at multiple secondary fixation points arranged radially around the primary fixation point. The FAS also receives feedback signals from the patient-operated feedback device in response to an activation thereof by the patient. The feedback signals are indicative of a location and possible size of the patient-perceived floater in the patient's field of vision. Additionally, the FAS calculates a numeric floater severity score using the feedback signals that quantifies the subjective severity of the floater(s).

### CROSS-REFERENCE TO RELATED APPLICATIONS

This application claims priority to and benefit of U.S. Provisional Patent Application No. 63/614,830 filed Dec. 26, 2023, which is hereby incorporated by reference in its entirety for all purposes.

### INTRODUCTION

The present disclosure relates to hardware and associated software-based techniques for assessing the severity of floaters in the vitreous body of a patient's eye.

The human eye is filled with a collagen-containing viscous gel referred to as the vitreous body, vitreous humor, or simply the vitreous. The vitreous body of a healthy eye is transparent, a characteristic that permits light entering the eye through the pupil to propagate, without obstruction, through the vitreous body to the retina. However, factors such as the patient's age, myopia level, and injury history can cause the vitreous body to liquify and collagen fibers to clump over time. Within the eye's vitreous cavity, the clumped collagen fibers may cast shadows on the retina. The shadows can manifest as irregularly shaped lines, cobwebs, or flecks and can appear anywhere within the patient's field of vision.

While floaters are often small or transparent enough for the patient to ignore, floaters may adversely obstruct the patient's vision to some extent depending on their size, quantity, and location within the vitreous chamber. In order to properly address severity and potentially treat symptomatic floaters, an eye surgeon may illuminate the vitreous chamber and view the vitreous body in real-time using a set of magnifying optics. However, floaters are dynamic phase objects that typically absorb just 1-2% of incident light. As a result of floater composition and large variations in floater tolerance across a given patient population, it can be difficult to determine floater severity in an accurate and repeatable manner.

### SUMMARY

Disclosed herein are a system and an associated office-based methodology for assessing the severity of floaters in a patient's eye. The present disclosure enables in-office performance of a simplified, technician-monitored, self-instructing psychophysical floater assessment according to standardized parameters. Among other attendant benefits, the present solutions enable accurate and repeatable assessment of floater severity to inform possible treatment decisions, for instance laser vitreolysis or vitrectomy surgery. In particular, the system and method as described herein may be used before interactive real-time visualization to help identify floaters that may be medically significant or symptomatic, i.e., candidates for possible surgical treatment.

In particular, a representative embodiment of a system for subjectively assessing the severity of patient-perceived floaters in an eye of a patient, with the system capturing the patient's responses to floaters perceived anywhere in the patient's field of vision. The system may include a display screen, a patient-operated feedback device, and an electronic floater assessment system (FAS) in communication with the display screen and the feedback device. The FAS is configured to perform a psychophysical floater assessment by presenting a visual target on the display screen, e.g., a viewing station or virtual reality (VR) glasses in different implementations. Target presentation occurs at a primary fixation point, which is typically a neutral “straight ahead” fixation as appreciated in the art.

In one or more optional implementations, multiple secondary fixation points may be arranged eccentrically at varying distances or locations around the primary fixation point, such that the secondary fixation points are all extrafoveal positions, for instance located above, below, left, right, or diagonally with respect to a visual axis extending between the patient's pupil to the primary fixation point.

The FAS in the embodiments contemplated herein receives feedback signals from the feedback device in response to an activation thereof by the patient, with the feedback signals being indicative of a location of the patient-perceived floater(s) relative to the visual target. The FAS thereafter calculates a numeric floater severity score using the feedback signals. The surgeon can thereafter attribute an actual medical significance to the floater(s) based on the patient's subjective input and other objective and/or subjective criteria as described below.

In one or more embodiments, the FAS is configured to present the visual target on the display screen at the primary fixation point, with the patient thereafter maintaining focus on the primary fixation point while recording instances of floaters within their field of vision. In other optional implementations the visual target may be sequentially presented above, to each side of, and below the primary focal point in accordance with a predetermined assessment sequence, with the visual target possibly returned to the location of the primary fixation point in between assessment at each of the secondary fixation points. Such an approach is based on the Busacca ascension phenomenon detected via a physician-performed slit lamp test, and is useful for collecting information about movement of the vitreous body with respect to eye movement, e.g., to determine if a floater consistently obstructs the patient's central vision.

In embodiments in which the optional secondary fixation points are implemented, the FAS may also be configured to maintain the visual target at the primary fixation point and each of the different secondary fixation points for a calibrated dwell time, e.g., several seconds or more at each point.

In a possible implementation, the different secondary fixation points may be separated from each other by about 5° to about 10°.

The FAS may also be configured to diagnose one or more of the patient-perceived floaters as being potentially “medically significant”, i.e., sufficiently obstructive of the patient's normal vision to warrant further evaluation and possible surgical treatment. Standardization is key to the performance of the disclosed assessment. The true/actual medical significance of a given floater or population thereof is ultimately determined by the surgeon by correlating psychophysical data from the FAS and the method as described herein with vitreous examination/visualization and vitreous imaging via optical coherence tomography (OCT) and/or scanning laser ophthalmoscopy (SLO). Such obstruction may be evaluated using a density metric as described herein. This action may occur based on or using the floater severity score, which would allow an accurate and repeatable assessment of an otherwise subjective or patient-centric determination.

In a possible approach, the FAS may be configured to calculate the floater severity score using the feedback signals by assigning different weights to the particular locations at which the patient perceives floaters while maintaining fixation on the primary fixation point. The different weights may include a highest weight for the primary fixation point, i.e., floaters perceived in the patient's central vision. Floaters perceived farther away from the primary fixation point are present in the patient's eccentric or extrafoveal positions/field of vision, and may be assigned a lower severity score as disclosed herein.

The visual target may include a grayscale visual target arranged on a white background. In other embodiments, the visual target may include a white target arranged on a black or grayscale background, with the background possibly being varied during the assessment.

In some instances, the FAS is configured to dynamically transition the visual target between the primary fixation point and the different secondary fixation points with smooth pursuit, i.e., such that the visual target does not dwell at the primary fixation point or any of the different secondary fixation points, e.g., for more than about 1 s.

The FAS may be configured to perform the assessment in accordance with a particular set of visual characteristics of the visual target, e.g., a predetermined contrast level of the visual target to allow for specific eye types (normal eye versus one having cataracts). A technician interface device may be used to select and record the patient-appropriate visual characteristics.

The above-described features and advantages and other possible features and advantages of the present disclosure will be apparent from the following detailed description of the best modes for carrying out the disclosure when taken in connection with the accompanying drawings.

### BRIEF DESCRIPTION OF THE DRAWINGS

FIG. 1 is an illustration of an automated floater assessment system (FAS) for assessing floater severity in a patient's eye in accordance with the present disclosure.

FIG. 2 depicts an eye having a population of floaters, the severity of which may be subjectively assessed via the system illustrated in FIG. 1.

FIG. 3 is an illustration of a representative visual target in accordance with an aspect of the disclosure.

FIG. 4 is a flow chart describing a method for subjectively assessing floaters in a patient's eye using the representative system of FIG. 1.

FIG. 5 is an illustration of a representative visual target in accordance with another aspect of the disclosure.

The appended drawings are not necessarily to scale, and may present a somewhat simplified representation of various features of the present disclosure, including, for example, specific dimensions, orientations, locations, and shapes. Details associated with such features will be determined in part by the particular intended application and use environment.

### DETAILED DESCRIPTION

The present disclosure is susceptible of embodiment in many different forms. Representative examples of the disclosure are shown in the drawings and described herein in detail as non-limiting examples of the disclosed principles. To that end, elements and limitations described in the Abstract, Introduction, Summary, and Detailed Description sections, but not explicitly set forth in the claims, should not be incorporated into the claims, singly or collectively, by implication, inference, or otherwise.

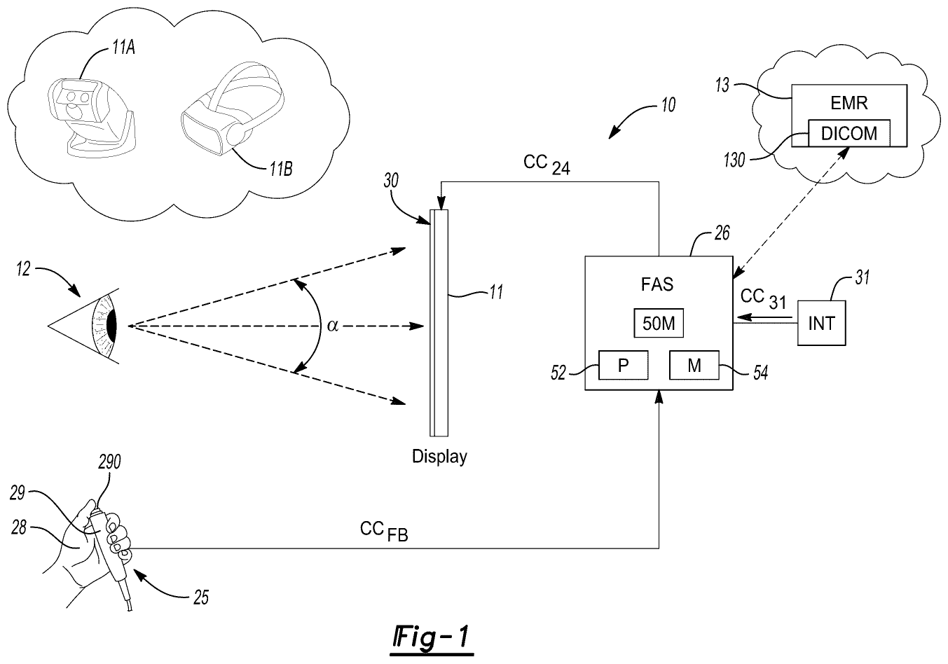

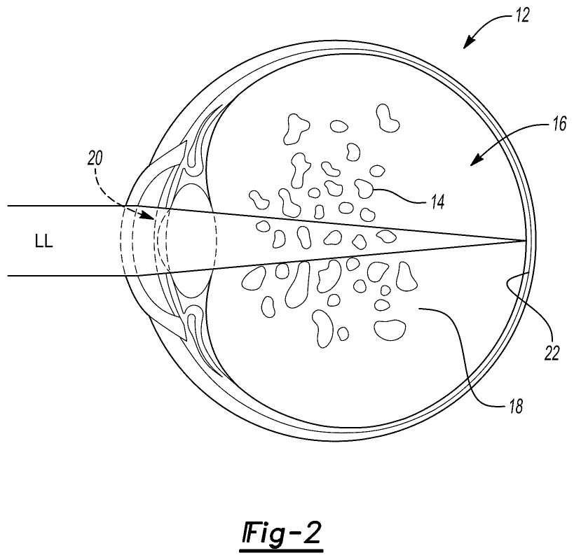

Referring to the drawings, wherein like reference numbers refer to like features throughout the several views, FIG. 1 depicts an interactive system 10 for subjectively assessing the severity of floaters within a patient's eye 12. As shown in FIG. 2, for instance, factors such as age, genetics, high myopia, or injury may result in discrete collections or amorphous clumps of collagen fibers within the vitreous chamber 16 of the eye 12. Such clumps are floating within the otherwise transparent vitreous body 18, and thus move with restrictions within the vitreous chamber 16 depending on their type and location.

When incident light (LL) passes through the pupil 20, any of the collagen fibers of the floaters 14 disposed in the path of the light (LL) may cast a shadow on the retina 22 located on the posterior wall of the vitreous chamber 16. As a result, the patient may perceive the shadows as visual disturbances or “floaters” as the collagen fibers move within the vitreous body 18, e.g., in response to eye movement, eye position, or gravitational settling. For illustrative clarity and simplicity, the particular collagen fibers casting shadows on the retina 22 and perceived by the patient are referred to hereinafter as floaters 14.

Floater severity is largely patient-specific and highly subjective. That is, a given size and/or location of the floaters 14 may be perceived or tolerated differently by different patients. The system 10 of FIG. 1 as contemplated herein thus enables the subjective in-office assessment of severity of the floaters 14 informed by real-time input from the patient rather than exclusively relying on objective measurements. Among other possible benefits, the present solutions allows a surgeon to determine, on a patient-by-patient basis, whether floaters 14 within the eye 12 are medically significant relative to an objective threshold, thus possibly warranting further investigation and possible surgical intervention, e.g., laser vitreolysis or vitrectomy surgery.

Referring once again to FIG. 1, the system 10 is operable for assessing severity of floaters 14 in the eye 12 of a patient 28, with a hand of the patient 28 shown for illustrative simplicity. The system 10 includes one or more display screens 11, a patient-operated feedback device 25, and an electronic floater assessment system (FAS) 26 in communication with the display screen(s) 11 and the feedback device 25. While shown as a representative display panel for simplicity, the display 11 may be constructed as a viewing station 11A, e.g., similar to driver license vision testing machines, auto-refractors, fundus cameras, or optical coherence tomography (OCT) packaging, etc., a set of virtual reality (VR) glasses 11B, digital oculars, goggles, or another device capable of presenting a visual target 30 to the patient 28 during an in-office floater assessment. During such a procedure, the FAS 26 receives electronic feedback signals (arrow CC_{FB}) from the feedback device 25 in response to the patient's activation thereof.

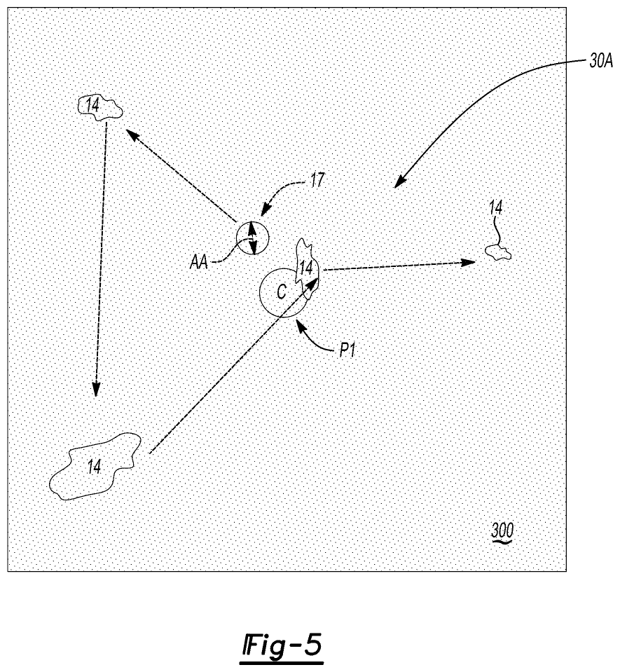

Referring briefly to FIG. 5, in an envisioned use scenario the patient 28 of FIG. 1 observes a visual target 30A at straight-ahead primary fixation point P1, which is also labeled “C” to indicate location in the patient's central vision. The visual target 30 of FIG. 1 may be displayed at different areas on the display screen 11 in other embodiments, with this option indicated by a viewing angle (α) in FIG. 1. Fixation and possible eye movement is verbally and/or visually queued, e.g., in response to voice prompts from the technician, e.g., “stare at the visual target without moving your eye”, or FAS-controlled movement of the visual target 30. The patient 28 activates the feedback device 25 when the patient 28 perceives a floater 14 while viewing the visual target 30 and indicates the relative location(s) (and possibly the size) of the floater(s) 14 relative to the primary fixation point P1

When the feedback device 25 of FIG. 1 is configured as a handheld multi-axis joystick 29 with one or more push buttons 290 as shown, the patient 28 may hold down the push button 290 while moving the joystick 29. The joystick may be connected to a base (not shown) in some configurations. Movement of the joystick 29 causes a patient-visible cursor 17 to move on the display screen 11, as indicated by the various dashed arrows in FIG. 5. Although the cursor 17 may be displayed in a range of possible shapes, a circular cursor 17 as shown may be used in one or more embodiments. When the cursor 17 overlaps a portion of the floater(s) 14, once again as perceived by the patient 28 of FIG. 1 while maintaining focus on the commanded primary fixation point P1 of FIG. 5, the patient 28 may double-click or otherwise activate the push button 290 to record the corresponding location of the floater 14. If more than one floater 14 is perceived, the patient 28 may repeat the sequence by steering the cursor 17 around the display screen 11 and registering all perceptible floaters 14 until the corresponding locations of such floaters 14 have been recorded.

In some implementations, the patient 28 may communicate the size of the floater 14 in addition to its location, e.g., by holding down the push button 290 after a single click, double-clicking, or providing other suitable control input and adjusting the diameter of the circular cursor 17, as indicated by double-headed arrow AA, and/or tracing the approximate perimeter of the floater 14. Alternatively, the patient 28 may tap a touch screen (not shown) after positioning the cursor 17 to thereby cause the FAS 26 to count the floater 14, and hold down the touch to resize the cursor 17 when indicating the size of the floater 14. Other devices such as knobs or dials may be used in other implementations, provided the hardware is configured to allow the patient 28 to report the location and possible size of each perceived or symptomatic floater 14.

Using a suitable completion signal, such as an extended depression of the push button 290 or depression of another button (not shown) situated on the feedback device 25, the FAS 26 is informed that all symptomatic floaters 14 have been identified by the patient 28 and recorded in memory 54 of the FAS 26. Suitable configurations for the feedback device 25 should not require the patient 28 to look away from the visual target 30, but may possibly include devices such as tablet computers or other touch screens that the patient 28 could operate by touch/feel. The feedback signals (arrow CC_{FB}) are indicative of perception of the floater(s) 14 in the field of vision of the patient 28 and possible subjective floater-based obstruction of the visual target 30, i.e., as determined by the patient 28 in the course of undergoing an in-office severity assessment. The FAS 26 then calculates a floater severity score using the feedback signals (arrow CC_{FB}) as described below with particular reference to FIG. 4.

Referring again to FIG. 1, the FAS 26 is depicted schematically as a unitary computational node solely for illustrative clarity and simplicity. Implemented embodiments of the FAS 26 may include one or more networked computer devices each with one or more processor(s) (P) 52 and sufficient amounts of the above-noted memory (M) 54, the latter including a non-transitory (e.g., tangible) computer-readable storage medium on which is recorded or stored a set of computer-readable instructions. Such instructions embody the method 50M, an exemplary embodiment of which is shown in FIG. 4 and described below, with the instructions being readable and executable by the processor(s) 52 to perform the method 50M.

As part of the present method 50M, the FAS 26 transmits display control signals (arrow CC₁₁) to the display screen(s) 11 to control the appearance, position, motion, and other possible characteristics or parameters of the visual target 30 as presented thereon. A technician interface device (INT) 31 may be in wired or wireless communication with the FAS 26 in some implementations, with mode or parameters selections by the technician resulting in transmission of input signals (arrow CC₃₁) to the FAS 26 as set forth below. The memory 54 may take many forms, including but not limited to non-volatile media and volatile media. Instructions embodying the method 50M may be stored in the memory 54 and selectively executed by the processor(s) 52 to perform the various functions described below.

As will be appreciated by those skilled in the art, non-volatile computer readable storage media may include optical and/or magnetic disks or other persistent memory, while volatile media may include dynamic random-access memory (DRAM), static RAM, etc., any or all which may constitute part of the memory 54. The input/output (I/O) circuitry may be used to facilitate connection to and communication with various peripheral devices used during the surgery or visualization procedure. Other hardware not depicted in FIG. 1 but commonly used in the art may be included as part of the FAS 26, including but not limited to a local oscillator or high-speed clock, signal buffers, filters, amplifiers, etc.

DICOM/EMR: use of the FAS 26 and the corresponding method 50M in a medical office environment as contemplated herein involves test ordering from an electronic medical record (EMR) 13 or an electronic health record (EHR). To that end, a secure networked communication with a Digital Imaging and Communications in Medicine (DICOM) protocol stack 130 of the EMR 13 is performed, as opposed to local storage of image data and test results. As appreciated in the art, a DICOM protocol stack 130 is used in medical office environments for magnetic resonance imaging (MRI), computed tomography (CT), digital X-ray, positron emission tomography (PET) scan, ultrasound, etc. Using the DICOM protocol stack 130, collected imaging data, biometry, visual fields, test orders, test results, etc., are securely sent and received via bi-directional communication with one or more cloud servers, e.g., using a local area network (LAN) connection or another suitable set of communication nodes. This also helps with entering patient data such as name, medical record number from a dropdown list. Floater assessment tests that are performed in accordance with the method 50M as described herein are ordered by a clinician, typically via scribe/secretary/technician, using the DICOM component of the EMR 13. The assessment results/data are thereafter automatically loaded into the patient's EMR 13 via the DIACOM protocol stack 130 via the above-noted secure network connection.

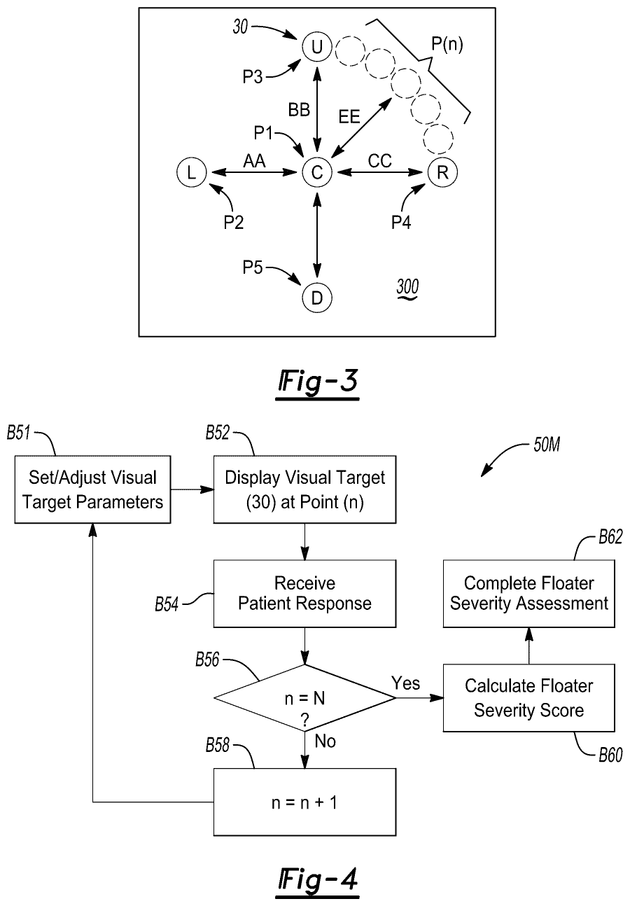

Referring now to FIG. 3 and as noted above, the floater assessment system (FAS) 26 shown schematically in FIG. 1 is configured to present the visual target 30 on the display screen 11. This action, which is accomplished via communication of the display control signals (arrow CC₁₁) of FIG. 1, occurs at the primary fixation point P1. Assessment may occur exclusively at the primary fixation point P1 as described above with reference to FIG. 5, or the patient 28 of FIG. 1 may also be prompted to view the visual target 30 at different secondary fixation points P2, P3, P4, P5, etc., each arranged radially around a primary fixation point P1 on the display screen 24. As noted above, use of the secondary fixation points P2, P3, P4, P5 collects additional information regarding movement of the vitreous body 18 with respect to movement of the eye 12 in a statistical manner.

The visual target 30 as contemplated herein may be a simple point of light on an appropriate background, or it may be a grayscale or black visual target 30 as shown and arranged on a white background 300. The visual target(s) 30 may be fixed at relatively low intensity (relative to the background 300) so as to not interfere with floater visualization. The background 300 could also be varied in two or more discrete steps for a density score in possible implementations. In other embodiments, a white target may be used, in which case the background 300 may be black or grayscale. Contemplated approaches include varying the background 300 from white to, e.g., sky blue, or shades of white approximating the appearance of a blank sheet of paper, a computer screen, or a white ceiling/wall. Initial variation may be performed during prototype validation, and thereafter via discrete steps to determine floater density when calculating the numeric floater severity score as noted herein. The particular black/grayscale and white combination may be calibrated a priori and used thereafter to perform a standardized assessment for a given population of patients, for instance those having an absence of cataracts and those having cataracts.

The primary fixation point P1 in the representative visual target 30 of FIG. 3, which may be located at an approximate center (C) of the display screen 11 (and the patient's central vision), forms a baseline “straight ahead” focal point. Relative to the primary fixation point P1, the optional secondary fixation point P2, P3, P4, and P5 are eccentrically positioned, e.g., to the left side (L), up/above (U), right side (R), and down/below (D) directions. The FAS 26 of FIG. 1 may present the visual target 30 on the display screen 11 at the primary fixation point P1 and possibly at the different fixation points P1-P5 according to a predetermined assessment sequence.

In the representative predetermined assessment sequence, the visual target 30 thus forms a software-controlled target on the display screen 11 of FIG. 1 that visually prompts the patient to focus in one or more directions relative to the primary fixation point P1. When testing fixation at different eye positions and possible effects on movement of the vitreous body 18, the patient 28 may be prompted to look upward to the secondary fixation point P3, back to the primary fixation point P1, left to the secondary fixation point P2, back to the primary fixation point P1, right to the secondary fixation point P4, back to the primary fixation point P1, down to the secondary fixation point P1P5, and back once again to the primary fixation point P1. The actual assessment sequence may be selected by the technician in one or more embodiments using the interface device 31 of FIG. 1, along with possible selection of the dwell time (or lack thereof) at the various fixation points P1-P5. In a likely implementation, however, all parameters for a given assessment will be standardized for a particular class of the eye 12 as noted above, e.g., one having cataracts or no cataracts, such that the technician need only to select the correct sequence for a particular patient.

The FAS 26 is thus configured to (i) maintain the visual target 30 at a particular focal point P1-P5 for the duration of the assessment, as in FIG. 5, or (ii) dynamically transition the visual target 30 between the primary/foveal fixation point P1 and the different secondary/extra-foveal fixation points P2-P5 as in FIG. 3 for determining motion of the vitreous body 18, such that the visual target 30 does not dwell at any of the fixation points P1-P5, e.g., for more than 1 second. As appreciated in the art, when the patient 28 blinks, the eye 12 of the patient 28 of FIG. 1 will roll upward, a response referred to as Bell's phenomenon. The eye 12 then refocuses upon completion of the blink. Motion sequences of the eye 12 are thus potentially beneficial for evaluating resulting motion of the vitreous body 18 and effects thereof on the resulting position of possible floaters 14 in the patient's field of view. The technician may elect to repeat the sequence(s), performing the assessment at least three times to acquire sufficient data for a statically meaningful assessment. Both (i) and (ii) may be performed in the course of a complete assessment to provide additional details as to the true state of floater-based obstruction at different fixations or combinations thereof.

In one or more implementations, the FAS 26 may maintain the visual target 30 at the primary fixation point P1 for the duration of the floater assessment (FIG. 5). Alternatively, when the different secondary fixation points P2-P5 are used (FIG. 3), such points may be separated from each other by about 5° to about 10°, or by another suitable angular value. One or more additional secondary fixation points P (n) may be used between the four nominal secondary fixation points P2-P5 in some embodiments, e.g., between fixation points P2 and P3, P3 and P4, P4 and P5, and/or P2 and P5. Likewise, the FAS 26 may be configured to maintain the visual target 30 at one or more of the different fixation points P1-P5 for a calibrated dwell time, e.g., more than about 1 second (1s) or about 1-5 s in possible embodiments, to enable dynamic floaters 14 to reach a steady state.

A surgeon, clinician, or technician may use the optional interface device 31 of FIG. 1 to select the appropriate assessment, e.g., for a normal eye 12 versus one having cataracts as noted above, and to thereby select one or more characteristics of the visual target 30 shown in FIG. 3. That is, while a separate cataract protocol defeats the standardization of the described assessment, the use of brightness and/or contrast could still be increased for cataract if performed in a standardized manner. The FAS 26 may configure the visual target 30 before the assessment in response to the input signals (arrow CC₃₁) from the interface device 31. Once the assessment has been selected, the FAS 26 may be configured to maintain the visual target 30 at the primary fixation point P1, or dynamically transition the visual target 30 between the primary fixation point P1 and the different secondary fixation points P2-P5 as noted above with smooth pursuit, i.e., such that the visual target 30 does not dwell at the primary fixation point P1 or any of the different secondary fixation points P2-P5 for more than about 1 s. Possible stepwise or continuous contrast adjustments may likewise be commanded by the technician (or standardized) via the interface device 31, or preprogrammed into memory 54 of the FAS 26, for optimal sensitivity in view of the patient's eye health, vision capabilities, or photosensitivity.

Referring now to FIG. 4, the method 50M is described herein using discrete code segments, algorithm sequences, or logic blocks for illustrative clarity. Execution of corresponding instructions for each of the constituent blocks of the method 50M by the processor(s) 52 of the floater assessment system (FAS) 26 illustrated in FIG. 1 ultimately causes performance of the described action. The method 50M as contemplated herein is performed one eye at a time, e.g., using an eye patch, mechanical shutter, or otherwise blocking the patient's vision for the non-tested eye. Method 50M may be performed in response to a test order that a surgeon or clinician enters via a patient's electronic medical record.

An exemplary implementation of the method 50M commences at block B51. Here, the technician may set target parameters for an ensuing subjective floater assessment. For example, the technician may select target parameters or a calibrated assessment sequence of the primary fixation point P1 and possibly one or more secondary fixation points, e.g., P2-P5, from a set of options presented via the interface device 31. Selection of the options results in generation of the display control signals (arrow CC₃₁). Target parameters as used herein may include the physical appearance and motion characteristics (if any) of the visual target 30 of FIG. 3, which are standardized as noted herein. Among other possible target parameters, the technician may select a profile having a particular black-on-white (grayscale-on-white), or white-on-black/grayscale scheme, contrast level, and a predetermined size, shape, and location of each foveal and optional extra-foveal fixation point P1-P5 of FIG. 3.

Additional target parameters may include the progression order or sequence that the patient will follow, if any. For instance, while optimal benefits are contemplated herein by recording locations of patient-perceived floaters 14 while the patient fixates solely on the primary fixation point P1, as in FIG. 5, some implementations may also assess vision obstruction when the patient 28 is prompted to look between the various secondary fixation points P1-P5. Target parameters may also include a particular contrast level of the visual target 30 and its background. In a likely implementation, however, the parameters may be predetermined for a given patient population, e.g., cataracts/no cataracts as noted above, with the technician simply selecting the relevant assessment sequence for the patient whose floater severity is being assessed.

In terms of adjustment, the technician may manually select the predetermined/standardized target parameters using the interface device 31. Example constructions may include a touch screen device on which the options are displayed, or a panel of buttons or other suitable mechanisms allowing the technician to physically select the desired target parameters. In one or more embodiments, the interface device 31 may be voice-activated such that the technician may utter a desired target parameter, with the FAS 26 of FIG. 1 setting the target parameter in response to the spoken phrase. In still other embodiments, one or more sequences and associated target parameters may be programmed into memory 54 of the FAS 26, such that the technician need only manually or verbally select a preprogrammed option to cause the target parameters to be adjusted. Audio and screen text is also useful for standardization, efficiency (technician time), and for reducing the required training/skill level needed for performing the assessment in a repeatable and consistent manner across a wide range of patients 28. The method 50M proceeds to block B52 when the target parameters have been initially set or subsequently adjusted.

At block B52, the method 50M includes displaying the visual target 30 at an initial fixation point (n). While any of the various fixation points P1-P5 of FIG. 3 may serve as the initial fixation point (n) in accordance with the predetermined or standardized subjective floater assessment, the initial fixation point (n) would typically be the primary/foveal fixation point P1 of FIG. 3, i.e., when the patient is looking straight ahead. The primary fixation point P1 may be the only fixation point used in the assessment in one or more embodiments, with optional transition between the primary focal point P1 and the various eccentric secondary focal points P2-P5 being possible in other implementations. The method 50M thereafter proceeds to block B54.

At block B54, after verbal prompting to do so by the technician, the patient stares at the initial fixation point (n) and observes the appearance of the visual target 30 and areas around it in the patient's field of vision. A possible auditory instruction to the patient could instruct the patient to “look straight ahead at the target, and if you see a floater anywhere, press the button and move the cursor to the floater”, with the cursor 17 and push button 290 illustrated in FIGS. 5 and 1, respectively. As the floaters 14 move into and out of the incident light (LL) of FIG. 2, for example, the patient may perceive darker, larger, or otherwise more pronounced floaters 14 at various locations in their field of vision relative to the primary focal point P1. The patient may activate the feedback device 25 of FIG. 1 when one or more floaters 14 are perceived anywhere in their vision field, such as by depressing the push button 290 in the non-limiting push button embodiment of the feedback device 25 to initially indicate the presence of floaters 14. When this occurs, the processor 52 of the FAS 26 registers a floater-based vision event, e.g., by recording a bit code indicative of the presence of the floater(s) 14.

The initial depression of the push button 290 may trigger a counter of the FAS 26 that allows the patient 28 sufficient time to record information describing the location of the floater(s) 14 in their field of view relative to the primary fixation point P1. Using the multi-axis joystick 29 of FIG. 1, for instance, the patient 28 may steer the cursor 17 (FIG. 5) on the display screen 11 until the cursor 17 overlaps the symptomatic floater(s) 14. The patient 28 thereafter activates the push button 290, which may initiate another timed window to allow the patient 28 to move the cursor 17 to the corresponding location of other floaters 14. A predetermined termination signal, such as an extended depression of the push button 290 of several seconds or actions equivalent to the familiar “double-click” of computer mouse operation, could be used to signal the FAS 26 that the locations of all floaters 14 in the patient's vision field have been recorded or registered in memory 54. The method 50M proceeds to block B56 after completion of block B54.

Block B56 includes determining, via the FAS 26 in optional embodiments in which testing is desired at other fixation points P2-P5, whether the fixation point (n) corresponding to the current counter value equals a predetermined final fixation point (N) in the above-noted progression sequence. As an example, the representative fixation points P1-P5 of FIG. 3 may set its secondary fixation point P5 as the final fixation point (N). With N=P5 in this particular example, block B58 may entail determining whether all constituent fixation points of the progression sequence have been performed. One of the parameters entered at block B51 may include performance of more than one assessment sequence, e.g., two, three, or four iterations thereof, and therefore block B56 may also include determining whether a required number of iterations has been completed. The method 50M proceeds to block B58 when an assessment has not yet been performed at the final fixation point (N), or if it has, whether this has occurred as many times as was specified at block B51. The method 50M proceeds in the alternative to block B60 when an assessment has been performed at the final fixation point, i.e., when n=N.

At block B58, the FAS 26 of FIG. 1 increments a counter having a register value that corresponds to the current fixation point. Thus, block B58 effectively includes advancing to the next fixation point (n+1) in the programmed or selected progression sequence. To implement block B56, the FAS 26 may increment a register value of an integer counter as one of its various hardware components. The method 50M thereafter returns to block B51.

At block B60, the method 50M includes calculating a numeric severity score indicative of floater severity, as perceived by the patient. As described above, throughout the process the FAS 26 receives the feedback signals (arrow CC_{FB}) from the feedback device 25 in response to its activation by the patient. As an example, despite the presence of the floaters 14 of FIG. 2, the patient's vision may not be obstructed to a significant degree (from the patient's perspective) when viewing the visual target 30 of FIG. 3 at primary fixation point P1. The patient may, for instance, move the aforementioned cursor to areas surrounding the primary fixation point P1 without recording subjective vision obstruction at the primary fixation point P1. The same floaters 14 may become symptomatic, however, when the patient is prompted to view the visual target 30 at other positions, e.g., the secondary fixation points P2 and/or P4. Should this transpire, the patient activates the feedback device 25 to generate the feedback signals (arrow CC_{FB}), which in turn would be recorded in memory 54 of the FAS 26. The recorded results during floater assessment therefore correspond to the particular ocular position of the eye 12 and prompted fixation point P1-P5 when the floater(s) 14 are perceived, i.e., the particular fixation point P1-P5 being viewed when the feedback signals (arrow CC_{FB}) were recorded, along with the location of the floaters 14 relative to the fixation point.

When calculating the numeric severity score, the FAS 26 of FIG. 1 may assign different weights to each of the fixation points. In this manner, the standardized assessment programmed into the FAS 26 may associate a higher or lower relative significance to a particular fixation point at which the patient perceives the floaters 14. For instance, for the representative five fixation points P1-P5 of FIG. 3, a numeric floater severity score (FS) may be calculated using a formula such as:

$$FS = {W}_{1} P 1 + {W}_{2} P 2 + {W}_{3} P 3 + {W}_{4} P 4 + {W}_{5} P 5$$

within which each respective one of the weights W₁, W₂, W₃, W₄, and W₅ may be preassigned or calibrated. The weights may be the same or different, with a 1 or 0 associated with the fixation points P1-P5 based on whether or not the patient recorded obstructed vision at that particular fixation point. The weight may be highest for the primary fixation point P1 in some implementations relative to extra-foveal fixation points, i.e., W₁>>W₂, W₃, W₄, and W₅. For instance, if the patient 28 records presence of a floater 14 directly on the primary fixation point P1 itself, with an example of this being depicted in FIG. 5, this event is given more weight than if the same patient 28 were to record a floater 14 at a distance away from the primary fixation point P1 (also shown in FIG. 5). Thus, in embodiments in which only the primary fixation point P1 is used for floater assessment, the above-noted secondary points P2-P5 are eccentric regions surrounding the primary fixation point P1.

In another implementation in which movement between the various fixation points P1-P5 is prompted, the standardized test may assess floater severity at the primary fixation point P1 at various stages of the evaluation. For example, the above formula may be expressed as:

$$FS = {W}_{1 A} P 1 A + {W}_{2} P 2 + {W}_{1 B} P 1 B + {W}_{3} P 3 + {W}_{1 C} P 1 C + {W}_{4} P 4 + {W}_{1 D} P 1 D + {W}_{5} P 5 + {W}_{1 E} P 1 E$$

where different weights W_{1A}, W_{1B}, . . . , W_{1E} are associated with each successive assessment at the primary fixation point P1.

For example, in keeping with the above example sequence during which the patient is visually and/or verbally prompted to focus on the primary (foveal) fixation point P1, then the secondary (extra-foveal) fixation point P2, then focus again on fixation point P1 before proceeding to fixation point P3, the initial portion of a full assessment sequence involves two fixations on the visual target 30 at the primary fixation point P1, in this case denoted as P1A and P1B for clarity. Fixation point P1A may be associated with weight W_{1A}, with the subsequent fixation at point P1B having its own associated weight W_{1B}, and so on. In this manner the surgeon may assess the effects of particular movements or movement combinations on resulting steady-state/straight ahead fixation.

In one or more embodiments, the patient-operated feedback device 25 may record more than a simple/discrete record of visual obstruction. For example, rather than implementing a simple push button 290 as shown in FIG. 1, the feedback device 25 may record different values based on the patient's perceived number and/or density of floaters 14 in their view. Thus, in addition to number, the feedback device 25 may be used to signal the density of the perceived floaters 14, e.g., using analog inputs such as dials or knobs as noted elsewhere herein. In such an implementation, the FAS 26 may calculate the numeric severity score as a composite score based on density, number, and location of the floaters 14 as reported by the patient 28. The method 50M then proceeds to block B62 after ascertaining the numeric floater severity score (FS).

Block B62 entails completing the assessment of floater severity using the numeric severity score (FS) determined at block B60. Block B60 may include generating, via the FAS 26 of FIG. 1 based on the numeric floater severity score, a digital output file that characterizes the floater-based obstruction as being symptomatic or potentially medically significant, with true medical significance ultimately determined by the surgeon/clinician. The surgeon may review such an auto-generated severity determination and verify whether the result is in accordance with severity as determined in the surgeon's professional judgment after objective verification. A possible control action associated with block B62 include outputting a digital report in which the FAS 26 characterizes the floaters 14 as being potentially medically significant or symptomatic based on the subjective assessment performed via method 50M. In response to such a report, the surgeon may perform additional visualization techniques and localization of symptomatic floaters 14, with the surgeon possibly treating the floaters 14 via vitrectomy surgery or laser vitreolysis as noted above, or using other invasive or non-invasive treatment options.

Embodiments of the present disclosure are described herein. The disclosed embodiments are merely examples, however, and thus other embodiments can take various and alternative forms. The drawings are not necessarily to scale. Some features could be exaggerated or minimized to show details of particular components. Therefore, specific structural and functional details disclosed herein are not to be interpreted as limiting, but merely as a representative basis for teaching one skilled in the art to variously employ the present disclosure.

Certain terminology may be used in the following description for the purpose of reference only, and thus are not intended to be limiting. For example, terms such as “above” and “below” refer to directions in the drawings to which reference is made. Terms such as “front,” “back,” “fore,” “aft,” “left,” “right,” “rear,” and “side” describe the orientation and/or location of portions of the components or elements within a consistent but arbitrary frame of reference which is made clear by reference to the text and the associated drawings describing the components or elements under discussion. Moreover, terms such as “first,” “second,” “third,” and so on may be used to describe separate components. Such terminology may include the words specifically mentioned above, derivatives thereof, and words of similar import.

The detailed description and the drawings are supportive and descriptive of the disclosure, but the scope of the disclosure is defined solely by the claims. While some of the best modes and other embodiments for carrying out the claimed disclosure have been described in detail, various alternative designs and embodiments exist for practicing the disclosure defined in the appended claims.

We claim:

1. An automated system for assessing a subjective severity of a patient-perceived floater in an eye of a patient, the system comprising:
a display screen;
a patient-operated feedback device; and
a floater assessment system (FAS) in communication with the display screen and the patient-operated feedback device, wherein the FAS is configured to:
present a visual target on the display screen at a primary fixation point;
receive feedback signals from the patient-operated feedback device in response to an activation of the patient-operated feedback device by the patient, the feedback signals being indicative of a location of the patient-perceived floater relative to the visual target; and
calculate a numeric floater severity score using the feedback signals, wherein the numeric floater severity score quantifies the subjective severity of the perceived floater.

2. The automated system of claim 1, wherein the FAS is configured to present the visual target on the display screen solely at the primary fixation point.

3. The automated system of claim 1, wherein the patient-operated feedback device includes a multi-axis joystick, and wherein the FAS is configured to display and move a cursor on the display screen in response to motion of the multi-axis joystick to indicate the location of the perceived floater relative to the visual target.

4. The automated system of claim 1, further comprising:
generating, via the FAS based on the numeric floater severity score, a digital output file that characterizes the patient-perceived floater as being potentially medically significant.

5. The automated system of claim 1, wherein the FAS is configured to sequentially present the visual target at multiple secondary fixation points located eccentrically with respect to primary fixation point, according to a predetermined assessment sequence during which the visual target is sequentially presented above, to each side of, and below the primary fixation point.

6. The automated system of claim 5, wherein the FAS is configured to calculate the numeric floater severity score using the feedback signals by assigning different weights to the primary fixation point and the one or more secondary fixation points.

7. The automated system of claim 6, wherein assigning the different weights to the primary fixation point and the one or more secondary fixation points includes assigning a highest weight to the primary fixation point.

8. The automated system of claim 5, wherein the FAS is configured to dynamically transition the visual target between the primary fixation point and the secondary fixation points such that the visual target does not dwell at the primary fixation point or any of the secondary fixation points for more than about 1 second.

9. The automated system of claim 1, wherein the visual target includes a grayscale target arranged on a white background.

10. The automated system of claim 1, further comprising:
an interface device, wherein the FAS is configured to select a visual characteristic or parameter of the visual target in response to input signals from the interface device.

11. The automated system of claim 10, wherein the characteristic or parameter of the visual target includes a contrast level of the visual target.

12. A method for assessing a subjective severity of a patient-perceived floater in an eye of a patient, the method comprising:
presenting a visual target on a display screen, via a processor of a floater assessment system (FAS), at a primary fixation point;
receiving feedback signals from a patient-operated feedback device via the processor in response to an activation of the patient-operated feedback device by the patient, the feedback signals being indicative of a patient-perceived floater in a field of vision of the patient; and
calculating a numeric floater severity score via the processor using the feedback signals that quantifies the subjective severity of the patient-perceived floater.

13. The method of claim 12, wherein sequentially presenting the visual target on the display screen at the primary fixation point includes periodically adjusting a location of the primary fixation point on the display screen.

14. The method of claim 12, wherein receiving feedback signals from the patient-operated feedback device includes receiving the feedback signals from a joystick and a push button device.

15. The method of claim 12, further comprising:
generating, via the FAS based on the numeric floater severity score, a digital output file that characterizes the patient-perceived floater.

16. The method of claim 12, wherein calculating the numeric floater severity score using the feedback signals includes assigning different weights to the floater when perceived at the primary fixation point relative to the floater when perceived at an extrafoveal position in the field of vision.

17. The method of claim 12, wherein presenting the visual target on the display screen includes presenting a grayscale target on a white background.

18. The method of claim 12, further comprising:
receiving input signals from an interface device via the FAS; and
adjust a contrast level of the visual target in response to the input signals from the interface device.

19. A computer-readable storage medium on which is recorded instructions for assessing a subjective severity of a patient-perceived floater in an eye of a patient, wherein execution of the instructions by a processor of a floater assessment system (FAS) causes the processor to:
present a grayscale visual target on a white background of via a display screen at a primary fixation point;
receive feedback signals from a patient-operated feedback device in response to an activation of the patient-operated feedback device by the patient, the feedback signals being indicative of the patient-perceived floater in a field of vision of the patient;
calculate a numeric floater severity score via the processor using the feedback signals that quantifies the subjective severity of the patient-perceived floater; and
generate, based on the numeric floater severity score, a digital output file that characterizes the patient-perceived floater as being potentially medically significant.

20. The computer-readable storage medium of claim 19, wherein execution of the instructions by the processor of the FAS causes the processor to:
assign different weights to the patient-perceived floater when perceived at the primary fixation point relative to the floater when perceived at an extrafoveal position in the field of vision.
